(19) United States
(12) Patent Application Publication
(10) Pub. No.: US 2012/0128654 A1
(43) Pub. Date: May 24, 2012

(54) Allantoin Administration for the Treatment of Neurodegenerative Disease and Neurotrauma

(75) Inventors: Brian T. Terpstra, Lowell, IN (US); Caryl E. Sortwell, Grand Rapids, MI (US)

(73) Assignee: UNIVERSITY OF CINCINNATI, Cincinnati, OH (US)

(21) Appl. No.: 13/265,584

(22) PCT Filed: Apr. 23, 2010
(86) PCT No.: PCT/US2010/032201

§ 371 Date: Feb. 6, 2012

Related U.S. Application Data
(60) Provisional application No. 61/172,019, filed on Apr. 23, 2009.

(51) Int. Cl. A61K 31/4166; A61P 25/16; A61P 25/00; A61P 25/14; A61K 31/616; A61K 31/445; A61K 38/18; A61K 31/57; A61K 38/49; A61P 25/28; A61K 31/426

(52) U.S. Cl. 424/94.64; 514/390; 514/367; 514/319; 514/7.7; 514/171; 514/161

(57) ABSTRACT

Methods for inhibiting the progression of neurodegenerative diseases and treating neurotrauma-induced damage and cerebrovascular disease are provided herein, the methods including the administration of a safe and effective amount of allantoin to a patient in need thereof. Also provided are pharmaceutical compositions including allantoin for the inhibition of the progression of neurodegenerative diseases and for the treatment of neurotrauma-induced damage and cerebrovascular disease.

### FEDERALLY SPONSORED RESEARCH OR DEVELOPMENT

The presently disclosed invention and its respective embodiments were made with U.S. Government support under Grant No. 1F31NS059270, awarded by the NIH. The government has certain rights in this invention.

The presently disclosed subject matter relates to the field of neurodegenerative disease and neurotrauma. Specifically, the present invention relates to methods and pharmaceutical compositions for inhibiting the progression of neurodegenerative diseases and treating neurotrauma-induced damage and cerebrovascular disease comprising the administration of allantoin.

Neurodegenerative diseases, such as Parkinson's disease, Alzheimer's disease, Huntington's disease, and others, affect millions of individuals worldwide. In the United States alone, nearly one million people are currently living with Parkinson's disease. No cure is presently known, although treatment options, including surgery and medications, are available to manage symptoms.

Parkinson's disease occurs when cells in the area of the brain called the substantia nigra begin to malfunction and die. Cells in the substantia nigra produce dopamine, a neurotransmitter involved in coordinating movement. When levels of dopamine in the brain decrease, the brain's capacity to initiate and control movement declines. Primary motor symptoms of Parkinson's disease include resting tremor, rigidity, bradykinesia (slowness of movement), akinesia (lack of movement), and postural instability.

Oxidative stress contributes to the cascade leading to cell death in the substantia nigra. However, oxidative stress is also a factor in the progression of other neurodegenerative diseases, including Alzheimer's disease, amyotrophic lateral sclerosis (ALS), Huntington's disease, and Freidreich's ataxia, as well as cerebrovascular disease such as stroke and certain neurotrauma-induced brain injuries. See Emerit, et al., Neurodegenerative diseases and oxidative stress, Biomedicine & Pharmacotherapy 58:39-46 (2004).

Recent studies have suggested the purine metabolite uric acid may play a role in cell death and the progression of Parkinson's disease. Peripheral adenosine, the precursor to inosine, has been shown to provide functional neuroprotection against striatal 6-OHDA infusion in Wistar rats. See Zafar et al., Protective effect of adenosine in rat model of Parkinson's disease: neurobehavioral and neurochemical evidences, J. Chem. Neuroanat. 26:143-51 (2003). Although this study did not include a measure of the structural integrity of the nigrostriatal pathway, peripheral inosine treatment in other models decreases apoptosis and preserves cell bodies following transection. In a rodent model of neonatal middle cerebral artery (MCA) occlusion, twice daily i.p. administration of inosine decreased Cytochrome C expression and TUNEL staining in the cortex and hippocampus (See Deng et al., Effects of inosine on neuronal apoptosis and the expression of cytochrome C mRNA following hypoxic-ischemic brain damage in neonatal rats, Zhongguo Dang Dai Er Ke Za Zhi 8:266-71 (2006)). Hou et al. have shown that repeated i.p. inosine administration every eight hours resulted in significant sparing of retinal ganglion cells following optical nerve transection in adult rats (Hou et al., Neuroprotective effect of inosine on axotimized retinal ganglion cells in adult rats, Invest. Ophthalmom. Vis. Sci. 45:662-67 (2004).

While these studies demonstrate the neuroprotective properties of peripheral adenosine and inosine treatment, the compound responsible for this effect has not been identified. Adenosine and inosine are rapidly and extensively metabolized in the periphery indicating that these purines, when administered systemically, may be broken down before reaching the CNS. The end product of purine metabolism in most mammals is allantoin. Allantoin has been demonstrated to have antioxidant properties in vivo (Guskov et al., Effect of allantoin on the activity of enzymes providing regulation of the ROS-dependent status of organism, Dokl Biochem. Biophys. 379:239-42 (2001)).

Given the seriousness and the prevalence of neurodegenerative diseases worldwide, a substantial need exists to develop additional methods and compositions for the treatment of neurodegenerative diseases, neurotrauma, and cerebrovascular diseases influenced by oxidative stress.

Methods and compositions for inhibiting progression of a neurodegenerative disease and treating neurotrauma-induced damage and cerebrovascular disease are provided herein.

In one embodiment, a method of inhibiting progression of a neurodegenerative disease is provided, the method comprising administering a safe and effective amount of allantoin or a pharmaceutically acceptable salt, ester, racemate, or enantiomer thereof, to a patient in need thereof.

In another embodiment, a pharmaceutical compositions for inhibiting progression of a neurodegenerative disease is provided, the composition comprising a safe and effective amount of allantoin or a pharmaceutically acceptable salt, ester, racemate, or enantiomer thereof; and at least one pharmaceutically acceptable excipient.

In another embodiment, a method of treating damage caused by neurotrauma or cerebrovascular disease is provided, the method comprising administering a safe and effective amount of allantoin or a pharmaceutically acceptable salt, ester, racemate, or enantiomer thereof, to a patient in need thereof.

In another embodiment, a pharmaceutical composition for treating damage caused by neurotrauma or cerebrovascular disease is provided, the composition comprising a safe and effective amount of allantoin or a pharmaceutically acceptable salt, ester, racemate, or enantiomer thereof; and at least one pharmaceutically acceptable excipient.

These and other objects, features, embodiments, and advantages will become apparent to those of ordinary skill in the art from a reading of the following detailed description and the appended claims.

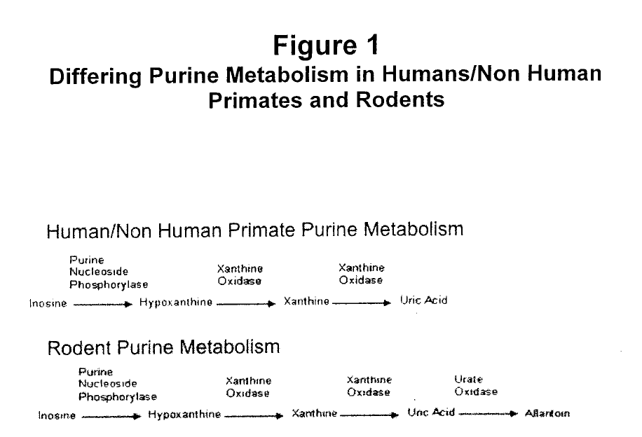

FIG. 1. Differing Purine Metabolism in Humans/Non-Human Primates and Rodents. Via evolution humans and non human primates have lost the activity of urate oxidase. Therefore, in humans and non human primates uric acid (UA) is the end product of enzymatic purine degradation. In rodents, UA is further metabolized to allantoin.

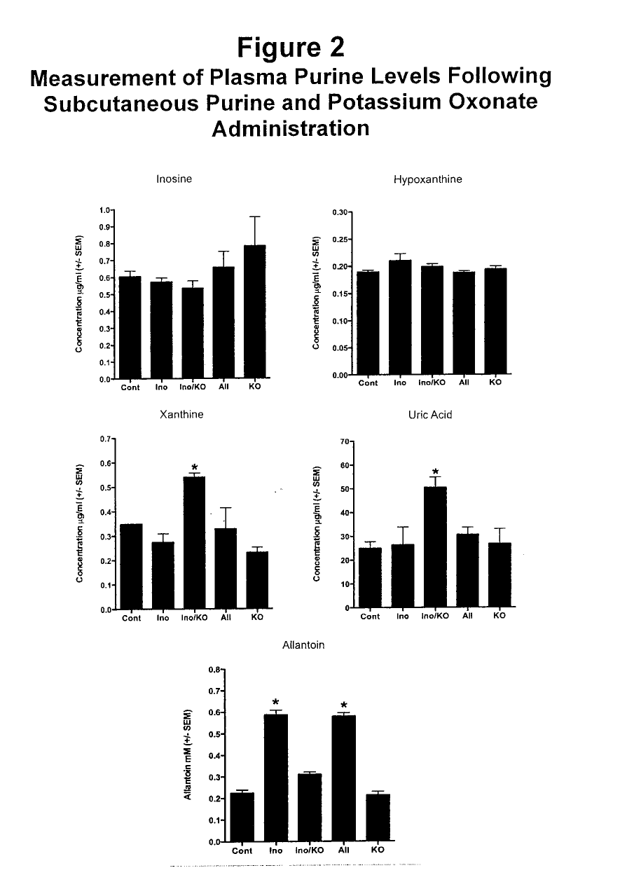

FIG. 2. Measurement of Plasma Purine Levels Following Subcutaneous Purine and Potassium Oxonate (KO) Administration. No pellet treatment impacted plasma levels of either Inosine or Hypoxanthine. Coadministration of Inosine and KO produced a significant elevation in plasma Xanthine and Uric Acid (p<0.01), treatments that did not yield neuroprotection. Allantoin levels were significantly increased by Inosine and Allantoin pellets (p<0.05), conditions in which neuroprotection was observed. All plasma samples were taken four days following pellet implantation. * indicates a significant difference from control values.

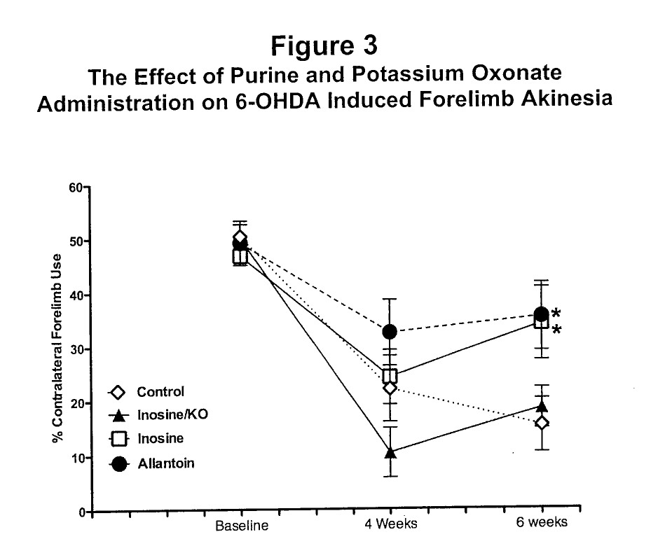

FIG. 3. The Effect of Purine and Potassium Oxonate Administration on 6-OHDA Induced Forelimb Akinesia. Subcutaneous inosine and allantoin administration significantly attenuated the forelimb akinesia induced by 6-OHDA infusion into the rat striatum (p=0.001). Additionally, preventing the conversion of uric acid to allantoin by coadministration of inosine and KO abolished inosine-mediated functional neuroprotection. * indicates a significant difference from control animals.

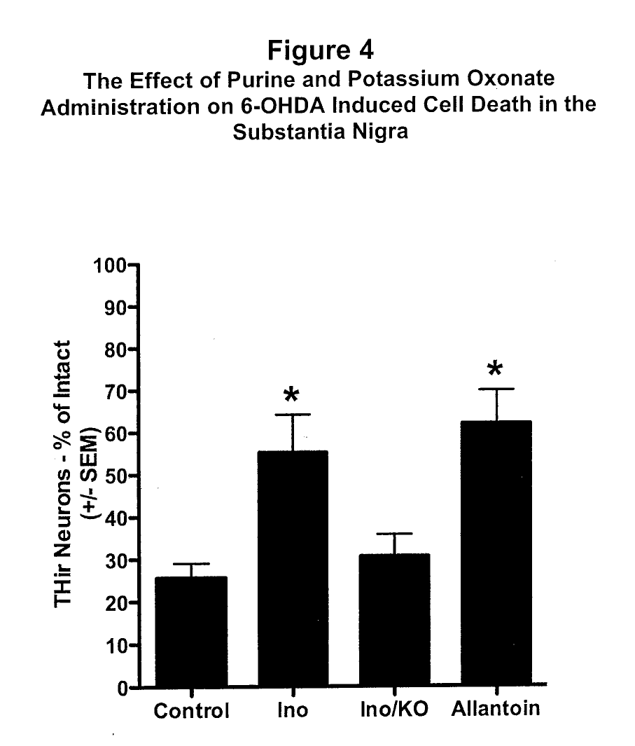

FIG. 4. The Effect of Purine and Potassium Oxonate Administration on 6-OHDA Induced Cell Death in the Substantia Nigra. Subcutaneous inosine (Ino) and allantoin treatment significantly protected nigral THir neurons against striatal 6-OHDA infusion (p=0.002), whereas preventing the breakdown of inosine to uric acid by potassium oxonate administration (Ino/KO) resulted in a similar lesion to control animals.

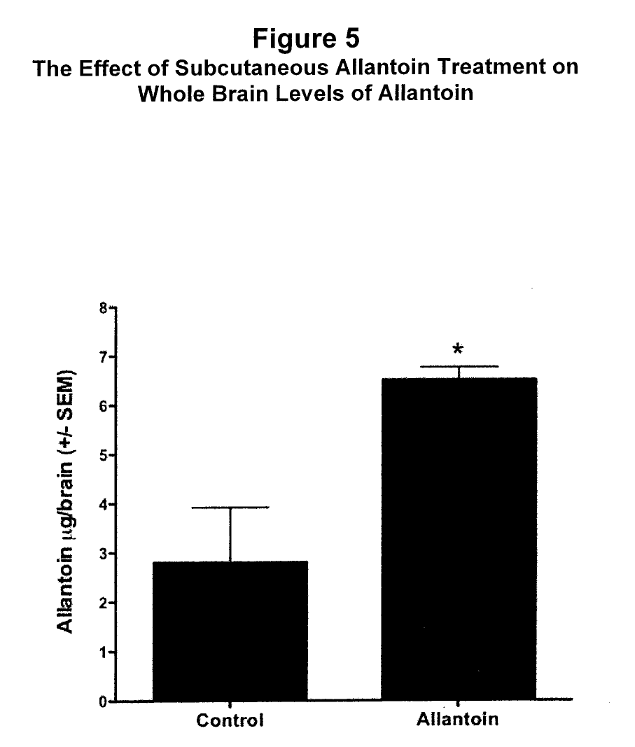

FIG. 5. The Effect of Subcutaneous Allantoin Treatment on Whole Brain Levels of Allantoin. Subcutaneous allantoin treatment resulted in a significant increase in whole brain allantoin levels three days following pellet implantation (p=0.008). * indicates a significant difference from control values.

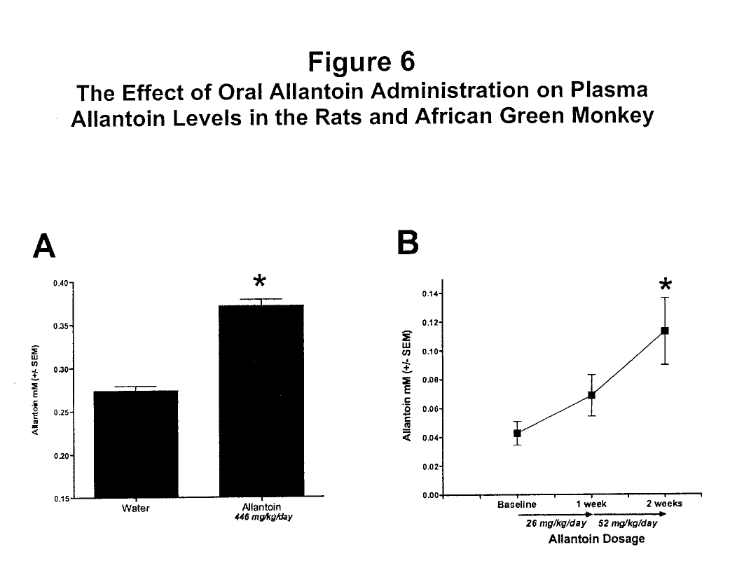

FIG. 6. The Effect of Oral Allantoin Administration on Plasma Allantoin levels in the rat and African Green Monkey. (A) Rat: Five consecutive days of 125 mg once daily oral allantoin administration resulted in a significant increase in plasma allantoin levels (*, p=0.001). (B) Monkey: Oral allantoin administration elevated plasma levels of allantoin, this elevation achieved significance after one week of daily feeding at 52 mg/kg/day dose (*, p<0.05, compared to baseline). This higher allantoin dose yielded an approximate tripling of baseline plasma allantoin levels in the African green monkey. * indicates a significant difference from control values.

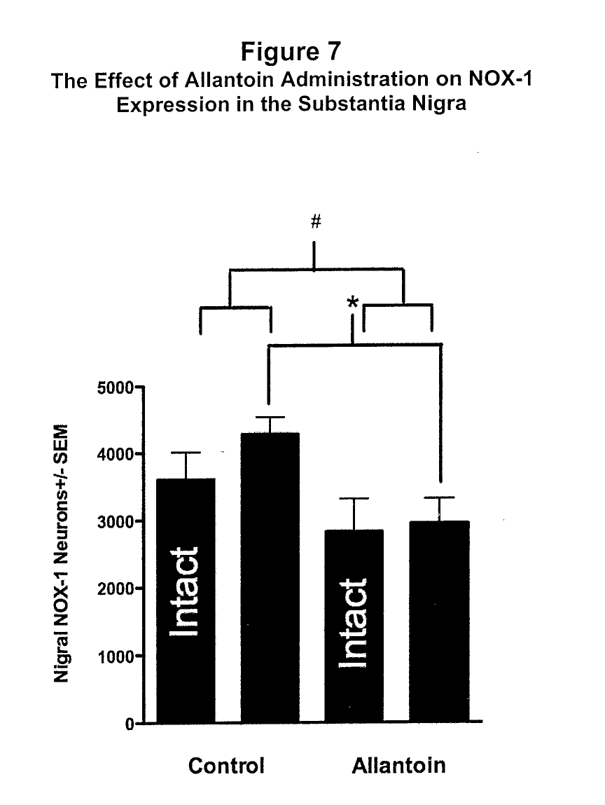

FIG. 7. The Effect of Allantoin Administration on NOX-1 Expression in the SN. Subcutaneous allantoin treatment resulted in a significant decrease in the number of NOX-1ir cells in both the 6-OHDA lesioned and intact SN relative to the 6-OHDA lesioned and intact SN in untreated controls (#, p<0.05). Further, allantoin treatment significantly reduced the NOX-1 expression induced by 6-OHDA (*, p<0.05).

The details of one or more embodiments of the presently-disclosed subject matter are set forth in this document. Modifications to embodiments described in this document, and other embodiments, will be evident to those of ordinary skill in the art after a study of the information provided in this document.

While the following terms are believed to be well understood by one of ordinary skill in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “neurodegenerative disease” refers to a disease characterized by a progressive decline in the structure, activity, and/or function of neural tissue, including brain tissue. Neurodegenerative diseases include, but are not limited to, Alzheimer's disease, Parkinson's disease, amyotrophic lateral sclerosis (ALS), Huntington's disease, Friedreich's ataxia, frontotemporal lobar degeneration, and dementia with Lewy bodies.

As used herein, the term “progression of a neurodegenerative disease” refers to the gradual worsening of the disease over time, whereby symptoms and neurochemical deficits become increasingly more debilitating and/or intense. Neurodegenerative disease progression often correlates to a decline in the structure, activity, and/or function of brain tissue.

As used herein, the term “inhibiting progression of a neurodegenerative disease” refers to slowing and/or stopping the progression of symptoms and neurochemical deficits of a neurodegenerative disease.

The term “treating,” as used herein, includes treatment of existing disease and prophylactic treatment of those at risk of developing the disease.

As used herein, the term “neurotrauma” refers to mechanical injury to the brain or spinal cord. The terms “damage caused by neurotrauma” or “neurotrauma-induced damage” refer to damage caused by a mechanical injury to the brain or spinal cord.

As used herein, the term “cerebrovascular disease” refers to brain dysfunctions related to disease of the blood vessels supplying the brain.

As used herein, the term “stroke” refers to the sudden death of brain cells due to a lack of oxygen when the blood flow to the brain is impaired by blockage or rupture of an artery to the brain.

As used herein, the term “allantoin” refers to the chemical compound having the formula C₄H₆N₄O₃ and the structure:

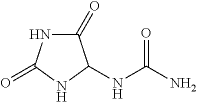

Allantoin is the product of oxidation of uric acid by purine catabolism in most mammals, excluding humans and higher apes. In humans, the metabolic pathway for conversion of uric acid to allantoin is not present (See FIG. 1).

The term “administering,” as used herein, refers to any route of administering a safe and effective amount of allantoin or a pharmaceutically acceptable salt, ester, racemate, or enantiomer thereof to a patient. In some embodiments, the administering include, but is not limited to, oral, intravenous, subcutaneous, and intramuscular administration.

The term “oxidative stress” refers to the steady state level of oxidative damage that occurs in a cell, tissue, or organ caused by reactive oxygen species. Oxidative damage occurs when a reactive compound (i.e. a compound having one unpaired electron) oxidizes a more stable compound by acquiring an electron from that compound. Oxidative stress is an influential factor in many diseases, including by not limited to, Parkinson's disease, Alzheimer's disease, amyotrophic lateral sclerosis (ALS), Huntington's disease, and Friedreich's disease, among many others. Oxidative stress is also a factor in neurotrauma-induced damage and cerebrovascular disease, including stroke.

Allantoin is the metabolic breakdown product of uric acid in some mammals. Humans, however, lack the enzyme required to metabolize uric acid to produce allantoin. The presently disclosed subject matter shows that inosine and allantoin treatment ameliorates forelimb akinesia and THir cell loss in the substantia nigra. However, when inosine is concurrently administered with KO, preventing the final step in the enzymatic metabolism of inosine (uric acid→allantoin; See FIG. 1), neuroprotection is not observed. Accordingly, the present disclosure is directed to methods and compositions for the treatment of neurodegenerative diseases and neurotrauma-induced damage comprising allantoin administration.

In one embodiment, a method of inhibiting progression of a neurodegenerative disease is provided, the method comprising administering a safe and effective amount of allantoin or a pharmaceutically acceptable salt, ester, racemate, or enantiomer thereof, to a patient in need thereof.

In certain embodiments, progression of the neurodegenerative disease is influenced by oxidative stress. In specific embodiments, the neurodegenerative disease is selected from the group consisting of Parkinson's disease, Alzheimer's Disease, amyotrophic lateral sclerosis (ALS), Huntington's disease, and Friedreich's ataxia. In a very specific embodiment, the neurodegenerative disease is Parkinson's disease.

In one embodiment of the present invention, administering comprises oral, intravenous, subcutaneous, and intramuscular administration. In another embodiment, the administering produces or yields a blood serum concentration of allantoin in a patient of from about 0.1 mM to about 5 mM.

In another embodiment, the method of inhibiting progression of a neurodegenerative disease further comprises administering a second active pharmaceutical ingredient effective for the treatment of the neurodegenerative disease.

In certain embodiments, the second active pharmaceutical ingredient is selected from the group consisting of rasagiline, levodopa, carbidopa, entacapone, ropinirole, pramipexole, donepezil, dopamine agonists, and catechol-O-methyl transferase (COMT) inhibitors, and combinations thereof. In a more specific embodiment, the second active pharmaceutical ingredient is selected from the group consisting of rasagiline, levodopa, carbidopa, entacapone, ropinirole, and pramipexole, for the treatment of Parkinson's disease. In another embodiment, the second active pharmaceutical is co-administered with allantoin.

In another embodiment, a pharmaceutical composition for inhibiting progression of a neurodegenerative disease is provided, the composition comprising a safe and effective amount of allantoin or a pharmaceutically acceptable salt, ester, racemate, or enantiomer thereof; and at least one pharmaceutically acceptable excipient. In certain embodiments, progression of the neurodegenerative disease is influenced by oxidative stress. In specific embodiments, the neurodegenerative disease is selected from the group consisting of Parkinson's disease, Alzheimer's Disease, amyotrophic lateral sclerosis (ALS), Huntington's disease, and Friedreich's ataxia. In a more specific embodiment, the neurodegenerative disease is Parkinson's disease.

The term “excipient,” as used herein, refers to any inactive substance incorporated into a pharmaceutical composition as a carrier for an active pharmaceutical ingredient. In one embodiment, the at least one pharmaceutically acceptable excipient is selected from the group consisting of polymers, resins, plasticizers, fillers, lubricants, diluents, solvents, co-solvents, buffer systems, surfactants, preservatives, sweetening agents, flavoring agents, pharmaceutical grade dyes or pigments, viscosity agents and combinations thereof. Suitable pharmaceutical excipients are well-known in the art. See, for example, Handbook of Pharmaceutical Excipients, Sixth Edition, edited by Raymond C. Rowe (2009). Further, the skilled artisan will appreciate that certain excipients may be more desirable or suitable for certain modes of administration of an active ingredient. It is within the purview of the skilled artisan to select the appropriate excipients for a given pharmaceutical composition.

In one embodiment, the pharmaceutical composition is an oral dosage form, such as a pill, tablet, capsule, or gel-filled capsule. In other embodiments, the dosage form can be a drink or syrup, an aerosol or inhaler, a liquid injection for intramuscular, intravenous, or subcutaneous injection, or a powder.

In another embodiment, the pharmaceutical composition further comprises a second active pharmaceutical ingredient. In certain embodiments, the second active pharmaceutical ingredient is selected from the group consisting of rasagiline, levodopa, carbidopa, entacapone, ropinirole, pramipexole, donepezil, dopamine agonists, and catechol-O-methyl transferase (COMT) inhibitors. In a specific embodiment, the second active pharmaceutical ingredient is selected from the group consisting of rasagiline, levodopa, carbidopa, entacapone, ropinirole, pramipexole, for the treatment of Parkinson's disease.

In another embodiment, a method of treating damage caused by neurotrauma or cerebrovascular disease is provided, the method comprising administering a safe and effective amount of allantoin or a pharmaceutically acceptable salt, ester, racemate, or enantiomer thereof, to a patient in need thereof. In certain embodiments, the damage caused by neurotrauma or cerebrovascular disease is influenced by oxidative stress.

In another embodiment, administering comprises oral, intravenous, subcutaneous, and intramuscular administration. In a specific embodiment, the administering produces or yields a blood serum concentration of allantoin in a patient of from about 0.1 mM to about 5 mM.

In another embodiment, the method of treating damage caused by neurotrauma or cerebrovascular disease further comprises administering a second active pharmaceutical ingredient effective for the treatment of the neurodegenerative disease or cerebrovascular disease. In a specific embodiment, the second active pharmaceutical ingredient is selected from the group consisting of anti-inflammatory drugs, erythropoietin, and progesterone. In another specific embodiment, the second active pharmaceutical ingredient is selected from the group consisting of tissue plasminogen activator (tPA), warfarin, and aspirin. In another specific embodiment, the second active pharmaceutical ingredient is co-administered with allantoin.

In another embodiment of the present invention, a pharmaceutical composition for treating damage caused by neurotrauma or cerebrovascular disease is provided, the composition comprising a safe and effective amount of allantoin or a pharmaceutically acceptable salt, ester, racemate, or enantiomer thereof and at least one pharmaceutically acceptable excipient. In a specific embodiment, the damage caused by neurotrauma or cerebrovascular disease is influenced by oxidative stress.

As with other embodiments of the present invention, the at least one pharmaceutically acceptable excipient is selected from the group consisting of polymers, resins, plasticizers, fillers, lubricants, diluents, solvents, co-solvents, buffer systems, surfactants, preservatives, sweetening agents, flavoring agents, pharmaceutical grade dyes or pigments, viscosity agents and combinations thereof. Suitable pharmaceutical excipients are well-known in the art. See, for example, Handbook of Pharmaceutical Excipients, Sixth Edition, edited by Raymond C. Rowe (2009). Further, the skilled artisan will appreciate that certain excipients may be more desirable or suitable for certain modes of administration of an active ingredient. It is within the purview of the skilled artisan to select the appropriate excipients for a given pharmaceutical composition.

In one embodiment, the pharmaceutical composition for the treatment of neurotrauma-induced damage or cerebrovascular disease is an oral dosage form, such as a pill, tablet, capsule, or gel-filled capsule. In other embodiments, the dosage form can be a drink or syrup, an aerosol or inhaler, a liquid injection for intramuscular, intravenous, or subcutaneous injection, or a powder.

In a specific embodiment, the pharmaceutical composition further comprises a second active pharmaceutical ingredient. In a specific embodiment, the second active pharmaceutical ingredient is selected from the group consisting of anti-inflammatory drugs, erythropoietin, and progesterone. In another specific embodiment, the second active pharmaceutical ingredient is selected from the group consisting of tissue plasminogen activator (tPA), warfarin, and aspirin.

### Pharmaceutical Compositions

The compositions of the embodiments of the invention comprise a safe and effective amount of allantoin or a pharmaceutically acceptable salt, ester, racemate, or enantiomer thereof and at least one pharmaceutically-acceptable excipient. In certain embodiments, the pharmaceutical compositions further comprise a second active pharmaceutical ingredient.

A “safe and effective amount” of allantoin is an amount that is effective to inhibit progression of neurodegenerative disease or treat damage from neurotrauma or cerebrovascular disease in a subject, without undue adverse side effects (such as toxicity, irritation, or allergic response), commensurate with a reasonable risk/benefit ration when used in the manner of this invention. The specific safe and effective amount will vary with such factors as the particular condition begin treated, the physical condition of the patient, the duration of treatment, the nature of concurrent therapy, if any, the dosage form used, the excipient(s) employed, and the dosage regimen desired. In a specific embodiment, suitable dosage forms provide a blood serum concentration of allantoin in a patient of from about 0.1 mM to about 5 mM.

Pharmaceutical excipients are selected from the group consisting of polymers, resins, plasticizers, fillers, lubricants, diluents, solvents, co-solvents, buffer systems, surfactants, preservatives, sweetening agents, flavoring agents, pharmaceutical grade dyes or pigments, viscosity agents and combinations thereof. Suitable pharmaceutical excipients are well-known in the art. See, for example, Handbook of Pharmaceutical Excipients, Sixth Edition, edited by Raymond C. Rowe (2009).

The compositions of the invention may be provided in a variety of forms suitable for oral, intravenous, subcutaneous, intramuscular, intraperitoneal, sublingual, rectal, nasal, pulmonary, and transdermal administration. Further, the skilled artisan will appreciate that certain excipients may be more desirable or suitable for certain modes of administration of an active ingredient. It is within the purview of the skilled artisan to select the appropriate excipients for a given pharmaceutical composition and mode of administration.

Examples of suitable oral dosage forms include tablets, lozenges, aqueous or oily suspensions, dispersible powders or granules, emulsions, hard or soft capsules, syrups or elixirs. Compositions intended for oral use may be prepared according to any method known in the art for the manufacture of pharmaceutical compositions and such compositions may contain one or more excipients. Such compositions my be coated by conventional methods, typically with pH or time-dependent coatings, such that the active ingredient is released in the gastrointestinal tract in the vicinity of the desired application, or at various times to extend the desired action. Suitable coatings include, but are not limited to, one or more of cellulose acetate phthalate, polyvinylacetate phthalate, hydroxypropyl methyl cellulose phthalate, ethyl cellulose, Eudragit® coatings, waxes and shellac.

Other suitable dosage forms include suspensions or solutions suitable for intravenous, intramuscular, or subcutaneous injection.

### EXAMPLES

The following examples are given by way of illustration and are in no way intended to limit the scope of the present invention.

### Example 1

### Animals and Materials

Male, Sprague Dawley rats (Harlan, 200-250 g) were used in these studies. All animals were given food and water ad libitum and housed in reversed light-dark cycle conditions. Inosine (INO), hypoxanthine, xanthine, uric acid (UA) and allantoin (ALL) were obtained from Sigma Aldrich (St. Louis, Mo.). Subcutaneous (S.C.) matrix-driven pellets were custom made by Innovative Research of America (Sarasota, Fla.).

### Example 2

### Peripheral Administration of INO, KO and ALL

Continuous s.c. delivery of INO, KO and ALL was achieved by implanting matrix-driven pellets into the s.c. space. Animals were anesthetized with isofluorane and prepared for surgery using sterile procedures. A small incision was made posterior to the scapula and the pellet was placed in the subscapular space contralateral to the incision. This placement technique prevents migration of the pellet towards the incision, thereby reducing the incidents of rejection. Finally, the incision was closed using aseptic procedures. To determine the roles of UA and allantoin in inosine-mediated neuroprotection, animals were treated with the urate oxidase inhibitor KO. This approach has been widely used to prevent the enzymatic metabolism of UA to allantoin in other rodent models of disease.

Rats also received oral allantoin via gastric gavage. Rats were firmly restrained via grasping the loose skin of the neck and back to immobilize the head. While maintaining the rat in an upright position, the gavage needle (20 G×1½″) was passed through the side of the mouth and advanced into the esophagus by following the roof of the mouth and advanced further toward the stomach. After the needle was passed to the correct length, 125 mg of allantoin, dissolved in water or water, was injected. This procedure was repeated daily for five days.

To determine whether oral allantoin administration could increase plasma levels of allantoin in non-human primates, African green monkeys were fed allantoin (n=4). All monkeys were initially fed 26 mg/kg/day for 1 week, after which the allantoin concentration was increased to a level of 52 mg/kg/day for an additional week. The allantoin was mixed to the appropriate concentration in fruit juice and given to the monkeys to drink.

### Example 3

### Intrastriatal 6-OHDA Injections

Rats were anesthetized prior to surgery with Equi-Thesin (0.3 ml/100 g body weight i.p.; chloral hydrate 42.5 mg/ml+sodium pentobarbital 9.72 mg/ml), their heads shaved, and then they were placed in a stereotaxic frame. Their skin was swabbed with Betadyne followed by 70% ETOH. The scalp was then opened to expose the skull. Two 1 mm holes were drilled into the skull and rats were injected in two sites in the striatum with 6-OHDA (MP Biomedicals, Solon, Ohio; 5 μg/μl 6-OHDA in 0.2% ascorbic acid, 0.9% saline solution). The coordinates for these injections were AP −1.6 mm, ML +2.4 mm, DV −4.2 mm and AP −0.2 mm, +ML 2.6 mm, DV −7.0 mm. The needle was zeroed at the skull directly above the injection site in order to target the DV coordinate. For each injection, the needle was lowered slowly to the injection site and 1 minute elapsed before injection commenced, 6-OHDA was injected at 0.5 μl/minute and at the end of the injection the needle was held in place for an additional 2 minutes prior to retraction. The wound area was cleaned with Betadine solution and the scalp was closed with surgical wound clips. This lesion paradigm results in a progressive loss of TH phenotype and frank DA cell death.

### Example 4

### Cylinder Testing for Forepaw Akinesia

Non-drugged, spontaneous use of the forepaws was measured in rats as described by Schallert (Schallert, T., Behavioral tests for preclinical intervention assessment, NeuroRx 3:497-504 (2006)) prior to pellet and intrastriatal 6-OHDA and at both four weeks and six weeks after 6-OHDA. During the dark cycle rats were placed in a clear Plexiglas cylinder and behavior was videotaped until the animal produced 20 weight bearing paw placements on the side of the cylinder, or for 5 minutes, which ever occurred first. Videotapes were analyzed by a rater blinded to treatment. The number of times the rat used its left, right, or both paws for weight bearing in a given trial was determined and noted. Data was reported as the percentage of contralateral (to 6-OHDA or vehicle injection), impaired forelimb use: [(contralateral+½ both)/(ipsilateral+contralateral+both)]×100.

### Example 5

### Collection of Brain Tissue

Animals were deeply anesthetized (60 mg/kg, pentobarbital, i.p.) and perfused intracardially with warm 0.9% saline containing 1 ml/L 10,000 USP heparin followed by ice-cold 0.9% saline. For immunoshistochemistry brains were removed, post fixed for 24 hours in 4% paraformaldehyde and transferred to 30% sucrose in 0.1M PO₄ buffer. For determination of purine levels brains were immediately removed and flash frozen in 3-methyl butane. Brains were stored at −80° C. until analysis.

### Example 6

### Tyrosine Hydroxylase Immunohistochemistry for SN Neurons

Brains were frozen on dry ice and sectioned at a 40 μm thickness using a sliding microtome. Every sixth section through the SN was processed for labeling with antisera against TH. Following blocking in 10% normal goat serum, tissue was incubated in primary antisera directed against TH (Chemicon MAB318, mouse anti TH 1:4,000) overnight at room temperature. Following primary incubation, sections were incubated in biotinylated secondary antisera against mouse IgG (Chemicon AP124B, 1:400) followed by the Vector ABC detection kit employing horseradish peroxidase (Vector Laboratories, Burlingame, Calif.). Antibody labeling was visualized by exposure to 0.5 mg/ml 3,3′ diaminobenzidine (DAB) and 0.03% H₂O₂ in Tris buffer. Sections were mounted on subbed slides, dehydrated to xylene and coverslipped with Cytoseal (Richard-Allan Scientific, Waltham, Mass.).

### Example 7

### NOX-1 Immunohistochemistry

Brains were frozen on dry ice and sectioned at a 40 μm thickness using a sliding microtome. Every sixth section through the midbrain was for incubated 30 minutes in sodium citrate 10 mM at 80 degrees Celsius. Following blocking in 10% normal goat serum, tissue was incubated in primary antisera directed against NOX-1 (Santa Cruz Biotechnology, Santa Cruz, Calif., 1:1000). Following primary incubation, sections were incubated in biotinylated secondary antisera against rabbit IgG (Vector Laboratory, Burlingame, Calif., 1:200) followed by the Vector ABC detection kit employing horseradish peroxidase. Antibody labeling was visualized by exposure to 0.5 mg/ml 3,3′ diaminobenzidine (DAB) and 0.03% H₂O₂ in Tris buffer. Sections were mounted on subbed slides, dehydrated to xylene and coverslipped with Cytoseal.

### Example 8

### Stereology

Unbiased stereological counts of THir cells were performed using a BX52 Olympus microscope (Olympus America Inc.) interfaced with Microbrightfield stereological software and a Microfire CCD camera (Optronics, Goleta, Calif.). Utilizing 2 μm guard zones and a 50 μm×50 μm counting frame a grid size of 112 μm×183 μm was employed to assure that all coefficient of error values were ≦0.10. Using the optical fractionator principle, the regions of interest were individually outlined under a low magnification (1.25×). Using a 60× magnification lens with a 1.4 N/A the section thickness was empirically determined by first bringing the top of the section into focus and then using Microbrightfield software to step through the z-axis in 1 μm increments until the very bottom of the section was in focus. Once the top of the section was in focus, the z-plane was lowered at 1-2 μm intervals and cell counts were made according to stereological principles while focusing down through the z-axis.

### Example 9

### Quantification of NOX-1ir Cells

Absolute counts of NOX-1ir cells was performed using a BX52 Olympus microscope interfaced with Microbrightfield stereological software and a Microfire CCD camera. Counts of NOX1-ir neurons were made at 20× and the sum of these values was adjusted according to the method of Abercrombie (Abercrombie, M., Estimation of nuclear populations from microtome sections, Anat. Rec. 94:239-247 (1946)). To assure that no cells were excluded or counted more than once, each cell was marked utilizing StereoInvestigator 7.0 (Microbrightfield Williston, Vt.). The total number of markers recorded by the software was utilized to estimate the total number of NOX-1ir cells (Fawcettt, et al., Dopaminergic neuronal survival and the effects of bFGF in explant, three dimensional and monolayer cultures of embryonic rat ventral mesencephalon, Exp. Brain Res. 106:275-82 (1995)).

### Example 10

### Dissection of Striatum for HPLC Analysis

Frozen brains were held at −18° C. for at least one hour prior to dissection. 1-2 mm coronal slabs were blocked from each brain utilizing a brain blocker (Zivic, Pittsburg, Pa.) and striatal tissue from both hemispheres were microdissected while being held at a constant −12° C. on a cold plate (Teca, Chicago, Ill.). Frozen dissected structures were placed individually in vials and stored at −80° C. until analysis.

### Example 11

### High Performance Liquid Chromatography (HPLC)

For HPLC analysis of plasma inosine, hypoxanthine, xanthine and UA, an antioxidant solution was added to an equal volume of plasma and samples were centrifuged at 14,000 g for 20 minutes at 4° C. The supernatant was collected for HPLC analysis. Samples were separated on a Hypersil ODS column (Thermo-Fisher Scientific, Waltham, Mass.). Compounds were detected using a Photodiode Array Detector attached to a Waters 2695 Solvent Delivery System under the following conditions: the mobile phase consisted of 0.5% KH₂PO₄ at a pH of 4.6. Unknown samples were quantified against a 6-point standard curve with a minimum r² of 0.97. Quality control samples were interspersed with each run to ensure HPLC calibration. Data are expressed in μg/ml.

### Example 12

### Enzymatic Detection of Allantoin

For plasma analysis of allantoin venous blood was obtained from the right ventricle (rat) and collected from repeated blood draws (African green monkey). Blood was immediately centrifuged at 10,500 rpms (this needs to be in G) for 5 minutes. Supernatant was then removed and analyzed for allantoin. Enzymatic detection of allantoin was performed as previously described with the exception of reaction volumes being adjusted to accommodate the use of a 96 well microtiter plate (Muratsubaki et al., Enzymatic assay of allantoin in serum using allantoinase and allantoate amidohydrolase, Anal. Biochem. 359:161-166 (2006)). First, 0.675 units of allantoinase and 2.5 units of glutamate dehydrogenase were added to a solution consisting of 0.5 m Tris, 10 mM MnCl₂, 50 mM ADP and 50 mM alphaketoglutarate (stock solution). Fifty microliters of plasma was added to 195 μl of stock solution in order to hydrolyze allantoin in the sample resulting in the production of allantoate and ammonia. The ammonia produced by this reaction was eliminated by the glutamate dehydrogenase in the stock solution. Following a 15 minute incubation, absorbance was read on a spectrometer at 340 nm. In the second step, allantoate was then hydrolyzed by the addition of allantoate amidohydrolase (0.14 units in 5 μl of 50 mM Tris and 0.2 mM EDTA) resulting in the production of ureidoglycine and ammonia. The ammonia produced by this reaction was eliminated by the glutamate dehydrogenase in the stock solution. Following a 10 minute incubation absorbance was once again measured at 340 nm. The final absorbance was subtracted from the absorbance following the addition of allantoinase. All sample were compared to known standards.

To measure brain allantoin levels, whole brains were freeze dried, ground into a fine powder and homogenized in 0.8 ml of 2.5% trichloroacetic acid (TCA) per 100 mg. The homogenate was centrifuged at 18,000 rpm for 15 min. Supernatant was removed and added to 5 ml of diethyl ether and centrifuged at 2500 rpm for 3 minutes in order to remove TCA from the sample. Following centrifugation, the upper ether phase containing TCA was carefully discarded using a capillary glass pipette without contamination of lower water phase. This process was repeated three times. To remove ammonia, the sample was added to 500 mg of DOWEX and centrifuged at 400 rpms for five minutes. The remaining supernatant was then analyzed for allantoin.

### Example 13

### Statistical Analysis

Statistical comparisons in lesioned animals were analyzed by two-way repeated measures ANOVA followed by a Tukey post hoc test. For plasma and tissue levels of purines a one-way ANOVA followed by a Tukey post hoc test was utilized.

### Example 14

### Impact of Peripheral Purine Administration on Plasma Purine Levels

To determine the impact of peripheral administration of purines on plasma purine levels, animals were divided into 5 groups INO (n=12), INO/KO (n=12), ALL (n=12), KO (n=12), and sham pellet implantation. All drugs were administered via matrix driven S.C. pellets. Venous blood was taken from the right ventricle of half of the animals in each group 4 days following pellet surgery. This procedure was repeated to obtain blood from the remaining animals 7 days following pellet surgery. Blood was then processed for analysis of plasma levels of INO, hypoxanthine, xanthine, and UA via HPLC. Plasma ALL levels were determined enzymatically.

Plasma analysis showed that peripheral s.c INO treatment was able to significantly impact plasma purine levels four days following pellet implantation. Specifically, systemic s.c inosine treatment resulted in a 2-fold increase in plasma allantoin without affecting plasma levels of any other purines. When s.c. inosine was co-administered with KO, there was no increase in plasma allantoin; however, plasma xanthine and UA were both significantly elevated. Systemic s.c. allantoin administration resulted in an increase in plasma allantoin similar to what was seen in the inosine only group. FIG. 2 summarizes the impact of peripheral s.c. purine and KO administration on plasma purine levels. All plasma purine levels had returned to baseline seven days following pellet implantation.

### Example 15

### Comparison of the Neuroprotective Effects of INO and ALL

In the first experiment, rats were divided into four different groups INO (n=6), INO/potassium oxonate (KO blocks conversion of UA to ALL) (n=6), ALL (n=6), and sham pellet implantation. In the second experiment, two additional groups of rats were divided into a KO alone group (n=6) or control pellet group (n=6), All drugs were administered via matrix driven S.C. pellets. In all cases, pellet implantation surgery took place 3 days prior to intrastriatal 6-OHDA. In experiment 1, rats were assessed for forelimb akinesia prior to, and 3 and 6 weeks after intrastriatal 6-OHDA infusion. Rats were sacrificed via intracardial saline perfusion at either 6 (experiment 1) or 4 (experiment 2) weeks post-6-OHDA, postfixed in 0.4% paraformaldehyde and processed for unbiased stereology.

FIG. 3 shows the ability of systemic s.c. purine administration to significantly alter the effect of striatal 6-OHDA infusion on forelimb akinesia (F(3,23)=4.447, p=0.001). Inosine treatment significantly ameliorated forelimb akinesia six weeks post lesion. This effect was completely abolished when inosine was co-administered with KO. In the group receiving allantoin treatment, forelimb akinesia was reduced at four and six weeks post-lesion compared to the control group.

Systemic s.c. purine administration significantly reduced 6-OHDA induced THir cell death in the SN (F(3,22)=6.906, p=0.002). In animals treated with inosine the number of surviving THir neurons in the lesioned SN was 56%±9.0% relative to the intact side. However, when inosine was administered concurrently with KO, the number of surviving THir cells was similar to controls (33%±4.5% and 25%±3.4%, respectively). Allantoin treatment resulted in a level of protection similar to inosine treatment with 61%±7.9% of the THir cells remaining on the lesioned side. FIG. 4 illustrates the ability of systemic purine treatment to attenuate nigral DA cell death following striatal 6-OHDA infusion. Results from experiment 2 revealed no significant impact of KO treatment alone on the number of THir neurons in either the intact or lesioned SN (p>0.05, data not shown).

### Example 16

### Peripherally Administered Allantoin Crosses the Blood Brain Barrier

To determine the ability of allantoin to cross the intact blood brain barrier (BBB), non-lesioned rats received either allantoin pellets or sham implantation. Three days following pellet surgery, rats were sacrificed via intracardial saline perfusion and brains were immediately flash frozen. Whole brain levels of allantoin were determined enzymatically.

In non-lesioned rats, three days of systemic s.c. allantoin treatment significantly elevated whole brain allantoin levels (F(1,9)=11.37, p=0.008). Allantoin levels were approximately double in the brains of the allantoin treated group relative to control rats (see FIG. 5).

### Example 17

### Orally Administered Allantoin Increases Plasma Allantoin Levels in Rats and Monkeys

Rats received oral allantoin or water via gavage once daily for 5 days. Venous blood was obtained from the right ventricle two hours following the final dose. All monkeys were initially fed 26 mg/kg/day for 1 week, after which the allantoin concentration was increased to a level of 52 mg/kg/day for an additional week. Plasma was collected via repeated blood draw at baseline, at the end of Week 1 and at the end of Week 2. Plasma collection occurred an hour prior to the next feeding so that plasma levels of allantoin reflected the minimum level during the 24 hour period. Allantoin levels in plasma were determined utilizing the enzymatic detection assay. Neither the rats nor the monkeys exhibited any obvious negative health consequences from the allantoin treatment and the monkeys readily ate the allantoin, indicating that the drug was palatable. Oral administration of allantoin to the rats significantly increased plasma allantoin levels (F(1,8)=74.563, p<0.001) representing an approximate doubling of allantoin in the rat plasma. Similarly, oral allantoin administration to the monkeys elevated plasma levels of allantoin; the elevation achieved significance after one week of daily feeding at the higher 52 mg/kg/day dose (*, p<0.05, compared to baseline). This higher allantoin dose yielded an approximate tripling of baseline plasma allantoin levels in the African green monkey.

FIGS. 6A and B shows the effect of daily oral allantoin administration on plasma allantoin levels in both the rat and monkey.

### Example 18

### Effect of Peripheral ALL Treatment on Nigral NADPH-Oxidase-1 (NOX-1)

Increased NOX-1 expression is directly linked to increased oxidative stress (Vignais, P., The superoxide-generating NADPF oxidase: structural aspects and activation mechanism, Cell Mol. Life Sci. 59:1428-59 (2002)). To determine the effect of ALL on NOX-1 expression in the SN and Red Nucleus, animals received either ALL pellets (n=6) or sham implantation (n=6). Pellet implantation surgery took place 3 days prior to intrastriatal 6-OHDA infusion. Twenty-four hours post-lesion, animals were sacrificed via intracardial saline perfusion and the SN was processed for NOX-1 immunohistochemistry.

Subcutaneous delivery of ALL beginning 3 days prior to intrastriatal infusion of 6-OHDA decreased the number of NOX-1ir cells in the SN (F(1,7)=5.84, p=0.047). Twenty-four hours post-lesion the number of NOX-1ir neurons in the lesioned SN of the ALL was significantly reduced compared to controls (see FIG. 7).

All documents cited are incorporated herein by reference; the citation of any document is not to be construed as an admission that it is prior art with respect to the present invention.

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

1. A method of inhibiting progression of a neurodegenerative disease, the method comprising administering a safe and effective amount of allantoin or a pharmaceutically acceptable salt, ester, racemate, or enantiomer thereof, to a patient in need thereof.

2. The method of claim 1, wherein progression of the neurodegenerative disease is influenced by oxidative stress.

3. The method of claim 1, wherein the neurodegenerative disease is selected from the group consisting of Parkinson's disease, Alzheimer's Disease, amyotrophic lateral sclerosis (ALS), Huntington's disease, and Friedreich's ataxia.

4. The method of claim 3, wherein the neurodegenerative disease is Parkinson's disease.

5. The method of claim 1, wherein administering comprises oral, intravenous, subcutaneous, and intramuscular administration.

6. The method of claim 1, wherein the administering yields a blood serum concentration of allantoin in a patient of from about 0.1 mM to about 5 mM.

7. The method of claim 1, further comprising administering a second active pharmaceutical ingredient effective for the treatment of the neurodegenerative disease.

8. The method of claim 7, wherein the second active pharmaceutical ingredient is selected from the group consisting of rasagiline, levodopa, carbidopa, entacapone, ropinirole, pramipexole, donepezil, dopamine agonists, and catechol-O-methyl transferase (COMT) inhibitors.

9. The method of claim 7, wherein the second active pharmaceutical ingredient is co-administered with allantoin.

10. A pharmaceutical composition for inhibiting progression of a neurodegenerative disease comprising:
a safe and effective amount of allantoin or a pharmaceutically acceptable salt, ester, racemate, or enantiomer thereof; and
at least one pharmaceutically acceptable excipient.

11. The pharmaceutical composition of claim 10, wherein progression of the neurodegenerative disease is influenced by oxidative stress.

12. The pharmaceutical composition of claim 10, wherein the neurodegenerative disease is selected from the group consisting of Parkinson's disease, Alzheimer's Disease, amyotrophic lateral sclerosis (ALS), Huntington's disease, and Friedreich's ataxia.

13. The pharmaceutical composition of claim 12, wherein the neurodegenerative disease is Parkinson's disease.

14. The pharmaceutical composition of claim 10, wherein the at least one pharmaceutically acceptable excipient is selected from the group consisting of polymers, resins, plasticizers, fillers, lubricants, diluents, solvents, co-solvents, buffer systems, surfactants, preservatives, sweetening agents, flavoring agents, pharmaceutical grade dyes or pigments, viscosity agents and combinations thereof.

15. The pharmaceutical composition of claim 10, wherein the pharmaceutical composition is an oral dosage form.

16. The pharmaceutical composition of claim 10, further comprising a second active pharmaceutical ingredient.

17. The pharmaceutical composition of claim 16, wherein the second active pharmaceutical ingredient is selected from the group consisting of rasagiline, levodopa, carbidopa, entacapone, ropinirole, pramipexole, donepezil, dopamine agonists, and catechol-O-methyl transferase (COMT) inhibitors.

18. A method of treating damage caused by neurotrauma or cerebrovascular disease, the method comprising administering a safe and effective amount of allantoin or a pharmaceutically acceptable salt, ester, racemate, or enantiomer thereof, to a patient in need thereof.

19. The method of claim 18, wherein the damage caused by neurotrauma or cerebrovascular disease is influenced by oxidative stress.

20. The method of claim 18, wherein the cerebrovascular disease is stroke.

21. The method of claim 18, wherein administering comprises oral, intravenous, subcutaneous, and intramuscular administration.

22. The method of claim 18, wherein the administering yields a blood serum concentration of allantoin in a patient of from about 0.1 mM to about 5 mM.

23. The method of claim 18, further comprising administering a second active pharmaceutical ingredient effective for the treatment of the neurodegenerative disease.

24. The method of claim 23, wherein the second active pharmaceutical ingredient is selected from the group consisting of anti-inflammatory drugs, erythropoietin, progesterone, tissue plasminogen activator (tPA), warfarin, and aspirin.

25. The method of claim 23, wherein the second active pharmaceutical ingredient is co-administered with allantoin.

26. A pharmaceutical composition for treating damage caused by neurotrauma or cerebrovascular disease comprising:
a safe and effective amount of allantoin or a pharmaceutically acceptable salt, ester, racemate, or enantiomer thereof; and
at least one pharmaceutically acceptable excipient.

27. The pharmaceutical composition of claim 26, wherein the damage caused by neurotrauma or cerebrovascular disease is influenced by oxidative stress.

28. The pharmaceutical composition of claim 26, wherein the at least one pharmaceutically acceptable excipient is selected from the group consisting of polymers, resins, plasticizers, fillers, lubricants, diluents, solvents, co-solvents, buffer systems, surfactants, preservatives, sweetening agents, flavoring agents, pharmaceutical grade dyes or pigments, viscosity agents and combinations thereof.

29. The pharmaceutical composition of claim 26, wherein the pharmaceutical composition is an oral dosage form.

30. The pharmaceutical composition of claim 26, further comprising a second active pharmaceutical ingredient.

31. The pharmaceutical composition of claim 30, wherein the second active pharmaceutical ingredient is selected from the group consisting of anti-inflammatory drugs, erythropoietin, progesterone, tissue plasminogen activator (tPA), warfarin, and aspirin.
